(19) United States
(12) Patent Application Publication
(10) Pub. No.: US 2024/0257990 A1
(43) Pub. Date: Aug. 1, 2024

(54) LEAD APRON SUSPENSION SYSTEM AND METHOD

(71) Applicant: Stephen C. Miller, Charleston, SC (US)

(72) Inventor: Stephen C. Miller, Charleston, SC (US)

(21) Appl. No.: 18/379,391

(22) Filed: Oct. 12, 2023

Related U.S. Application Data
(60) Provisional application No. 63/415,385, filed on Oct. 12, 2022.

(51) Int. Cl. G21F 3/03; A61B 6/10

(52) U.S. Cl. CPC G21F 3/03; A61B 6/107

(57) ABSTRACT

An apparatus suspends a personal radiation protection garment above a work surface. The suspension apparatus comprises a support member positioned above the work surface, a cable mechanically suspended from the support member, and means for counter-balancing the weight of the personal radiation protection garment attached to the cable. Means for attaching the counter-balancing means to the personal radiation protection garment suspend the personal radiation protection garment. The attaching means includes means for disconnecting the garment from the counter-balancing means, the disconnecting means manually operable by one hand of a user and disposed within reach of the user while wearing the personal radiation protection garment attached to the counter-balancing means. The personal radiation protection garment is supported in suspension and may be disconnected from the counter-balancing means by the one hand of the user for releasing the personal radiation protection garment from suspension while still being worn by the user.

### CROSS REFERENCE TO RELATED APPLICATIONS

The present application claims priority to and benefit of U.S. Provisional Patent Application No. 63/415,385, filed on Oct. 12, 2022, the disclosure of which is hereby incorporated by reference herein in its entirety.

### BACKGROUND

A suspension system and method are described for medical equipment and instruments in a medical environment are described and, more particularly, a system and method are described for suspending a lead apron.

Radiation is used to perform many medical diagnostic and therapeutic tests and procedures, such as cardiac catheterizations. To minimize exposure to radiation, a medical operator commonly wears a protective garment containing radiation-absorbing materials, generally lead or other metals. The garment is worn in the fashion of a vest and apron, or a full lead jacket which reaches from the shoulder to the knees. These types of garments are uncomfortable, heavy and place significant stress on the operator's body, especially the spine. This may present a significant logistic and health challenge for medical operators in radiation environments, such as a catherization laboratory. Indeed, the effects of an operator wearing such a heavy protective garment over an extended period of time is known to be associated with diseases of the spine in the neck and back, knee disorders, and other musculoskeletal problems, which can result in disability, medical expenses, and decreased quality of life for the operator.

One solution is a suspension system for the heavy protective garments, such as the lead apron. A typical lead apron suspension system comprises an overhead rail, a trolley that runs on the rail, and a cable extending from the trolley. A personal radiation protection garment worn by the operator is suspended from the suspension system at the free end of the cable. The suspension system bears the weight of the garment for relieving the operator. Suspension systems and methods are described in U.S. Published Application No. 2017/0027245, filed Mar. 17, 2016, the contents of which is hereby incorporated herein by reference in their entirety.

Occasionally, an emergency may arise during a procedure which necessitates that the operator release the apron from the suspension system so as to allow an operator wearing the garment to move freely from the suspension system. The immediate result of which is, at release, the weight of the garment falls on the operator. This is a sudden impact on the body of the operator, which can cause many of the health problems the suspension system is designed to alleviate.

For the foregoing reasons, there is a need for a suspension system and method for use in a medical environment. In one aspect, the suspension system and method should allow an operator to wear a personal radiation protection garment for minimizing the weight on the body, while not substantially limiting freedom of movement. In another aspect, the suspension system should allow the operator to quickly release the garment from the suspension system without the deleterious effects of the weight of the garment falling on the operator.

### SUMMARY

An apparatus is provided for suspending a personal radiation protection garment above a work surface defining an area. The suspension apparatus comprises a support member adapted to be positioned above the work surface, a cable mechanically suspended from the support member, and means for counter-balancing the weight of the personal radiation protection garment, the counter-balancing means attached to the cable. Means for attaching the counter-balancing means to the personal radiation protection garment suspend the personal radiation protection garment. The attaching means includes means for disconnecting the garment from the counter-balancing means, the disconnecting means manually operable by one hand of a user and disposed within reach of the user while wearing the personal radiation protection garment attached to the counter-balancing means. The personal radiation protection garment is supported in suspension when attached to the counter-balancing means, and the personal radiation protection garment may be disconnected from the counter-balancing means by the one hand of the user for releasing the personal radiation protection garment from suspension while still being worn by the user.

### BRIEF DESCRIPTION OF THE DRAWINGS

For a more complete understanding of the suspension system and method, reference should now be had to the embodiments shown in the accompanying drawings and described below. In the drawings:

FIG. 1 is a perspective view of an embodiment of a suspension system for a personal radiation protection garment in use in a medical environment.

FIG. 2 is a front elevation view of the suspension system as shown in FIG. 1.

FIG. 3 is a side elevation view of the suspension system as shown in FIG. 1.

FIG. 4A is a front perspective view of an embodiment of a cushioning vest for use with the garment as shown in FIGS. 1-3.

FIG. 4B is a front perspective view of an embodiment of a hanger for use with the garment as shown in FIGS. 1-3.

FIG. 5 is a side elevation view of the personal radiation protection garment including the vest shown in FIG. 4 depicted in dashed lines.

FIG. 6 is a rear elevation view of an upper portion of the personal radiation protection garment including the vest depicted in dashed lines as shown in FIG. 5.

FIG. 7 is a front elevation cross-section view of a portion of the suspension system as shown in FIG. 1, including an embodiment of a hanger and a connecting system and a rear portion of the vest as shown in FIG. 4.

FIG. 8 is a rear perspective view of a portion of the suspension system as shown in FIG. 1, including the vest as shown in FIG. 4.

FIG. 9A is a front elevation view of the hanger and a portion of the suspension system as shown in FIG. 1, including an embodiment of means for connecting the hanger to the suspension system.

FIG. 9B is an exploded front elevation view of the hanger and the portion of the suspension system as shown in FIG. 9A.

### DESCRIPTION

Certain terminology is used in the following description for convenience only and is not limiting. The words “right”, “left”, “top”, and “bottom” designate directions in the drawings to which reference is made. The words “interior” and “exterior” refer to directions toward and away from, respectively, the geometric center of the core and designated parts thereof. The terminology includes the words above specifically mentioned, derivatives thereof and words of similar import.

A suspension system is described for use in suspending medical equipment and instruments and may be used with any conventional suspension system such as, for example, the system described by U.S. Pat. No. 7,608,847, issued Oct. 27, 2009, and entitled “System and Method for Implementing a Suspended Personal Radiation Protection System,” and the system described by U.S. Pat. No. 7,973,299, issued Jul. 5, 2011, and entitled “System and Method for Providing a Suspended Personal Radiation Protection System,” the contents of both of which are hereby incorporated by reference in their entirety. Accordingly, detailed explanations of the functioning of all of the components and method of such suspension system are deemed unnecessary for understanding of the present invention by one of ordinary skill in the art.

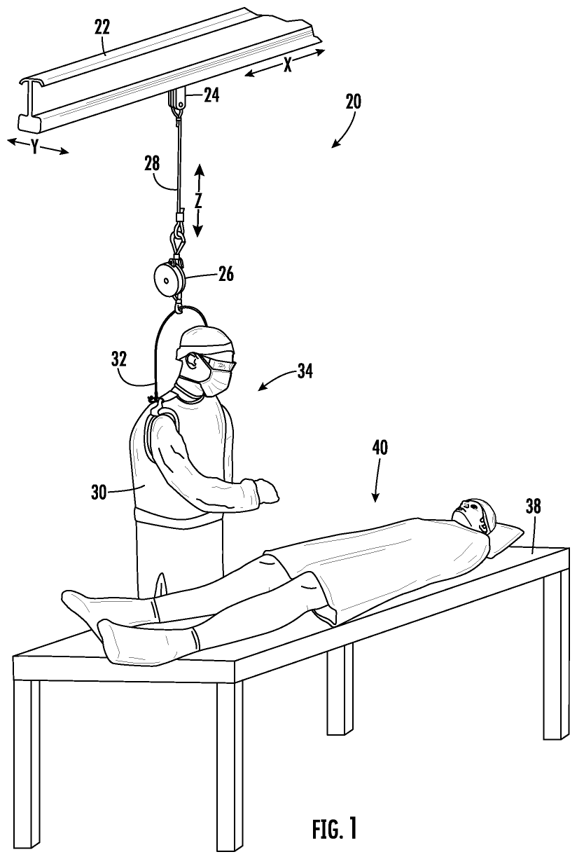

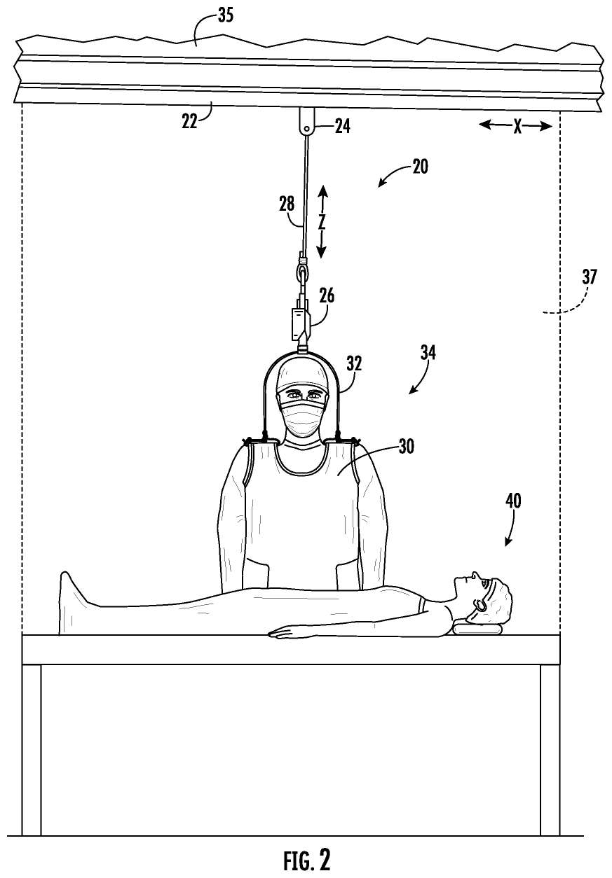

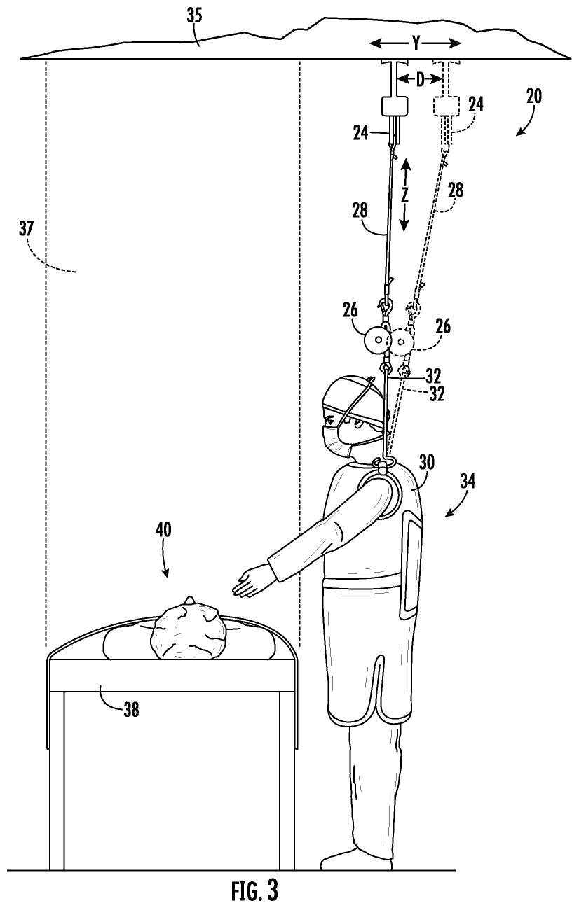

Referring now to the drawings, wherein like reference numbers refer to like elements in each of the several views, FIGS. 1-3 show a suspension system according to an embodiment of the present invention, which is generally designated at 20. The suspension system 20 comprises a rail 22, a trolley 24, a balancer 26 and a cable 28. A personal radiation protection garment 30 worn by an operator 34 is suspended from a hanger 32 which, in turn, is suspended from the suspension system 20. A radiation source (not shown) may be proximate to the operator 34. The radiation source may include any device emitting radiation. For example, in medical procedures, radiation sources may include x-ray machines, nuclear medicine, and devices used for radiation therapy. Other architectures and components of the suspension system 20 may be used without departing from the scope of the present invention.

The rail 22 may be affixed to any suitable supporting structure, in addition to or other than the ceiling 35, including a wall, floor, mobile structure, mobile frame, immobile frame, cable, or any other mechanical means for hanging an item over the head of an operator. While any supporting structure may be employed to mount the rail 22 thereto, the present description illustrates the rail 22 being affixed to the ceiling 35 as only an exemplary location to mount the rail 22. One skilled in the art would recognize that any other supporting structure may be substituted for the ceiling 35.

Referring to FIGS. 1-3, the rail 22 may be affixed to the ceiling 35 at any location. For example, the rail 22 may be secured to a portion of the ceiling 35 that is directly above a non-operating area, which may be any area that is not directly above an operating surface 38, such as a medical operating/surgery surface or patient examination surface. Preferably, the rail 22 is not placed directly over the operating surface 38. For example, the rail 22 may not be placed directly over where a patient 40 will be located during radiation treatments/operations.

The suspension system 20 is operable by any suitable means to allow free motion along a path defined by the rail 22 for the operator 34 wearing the suspended personal radiation protection garment 30. In the embodiment shown, the trolley 24 is configured to easily slide or roll along the rail 22 so that the operator 34 can move freely. The trolley 24 can attach via the cable 28 to the balancer 26, which suspends the personal radiation protection garment 30, such that the operator 34 can move freely in the X-spatial plane, defined by the X-spatial axis and the vertical Z-spatial axis. The X-spatial plane is designed to correspond to the operator's 34 desired work area. The operator 34 wearing the suspended personal radiation protection garment 30 may have a smooth and facile motion within this plane. The operator 34 is still allowed some movement along the Y-axis. For example, the operator 34 is allowed to bend forward or walk forward or backwards to the extent of the extension of the cable 28 attached to the counter balancer 26.

The cable 28 is operable to safely hold the amount of weight and force caused by the suspended personal radiation protection garment 30. In one preferred embodiment, the cable 28 may be suspended from the trolley 24 and may attach to the balancer 26. In another embodiment, the cable 28 may be suspended from the balancer 26 and may attach to the hanger 32. In yet another embodiment, the cable 28 may be suspended from the rail 22 and connect to the balancer 26. The cable 28 may be a strap, a rope or a belt. The cable 28 may be several feet long and allow the operator 34 to move extensively in the X-axis and extensively in the vertical Z-axis. The cable 28 also allows the operator 34 to move slightly outside the perimeter of the X-spatial plane. The cable 28 can include a swivel mount that permits free rotation of the cable suspension mechanism allowing the operator 34 to twist as needed. This may include a swivel hook or snap that connects the cable 28 to the balancer 26 or the rail 22.

As described above, the balancer 26 may be attached to the trolley 24 by the cable 28. The balancer 26 applies a constant and controllable uplifting force on the personal radiation protection garment 30. In one embodiment, the balancer 26 may be a spring balancer, which can include a coiled flat spring, similar to a clock spring, attached to a reel with a conical shape. The conical shape provides a variable mechanical advantage, which offsets the variance of the force provided by the spring as it winds or unwinds, such that there is a relatively constant force on the cable 28 within a definable working range. A suitable counter-balancer for use in an embodiment of the suspension system 20 is available from Packers Kromer of Omaha, Nebraska. Similar suitable counter-balancers are described in U.S. Pat. Nos. 6,042,087 and 7,424,997, the contents of both of which are hereby incorporated by reference in their entirety.

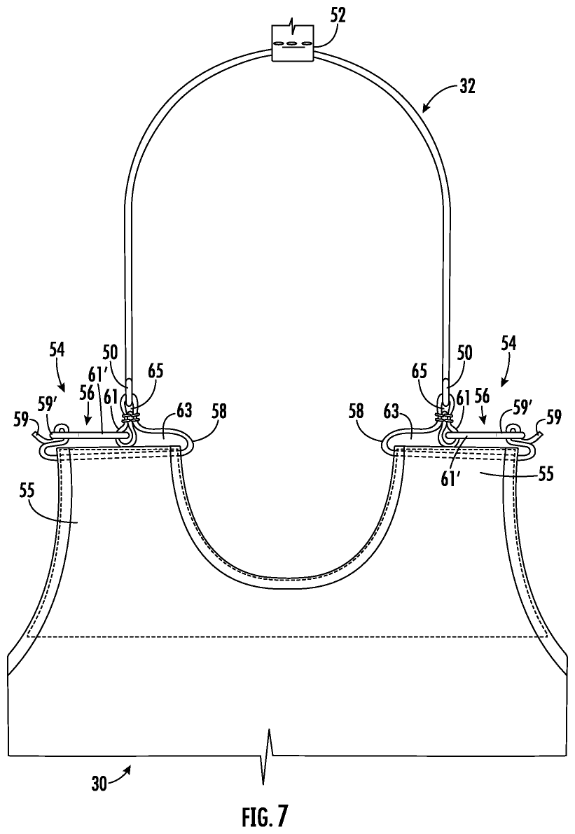

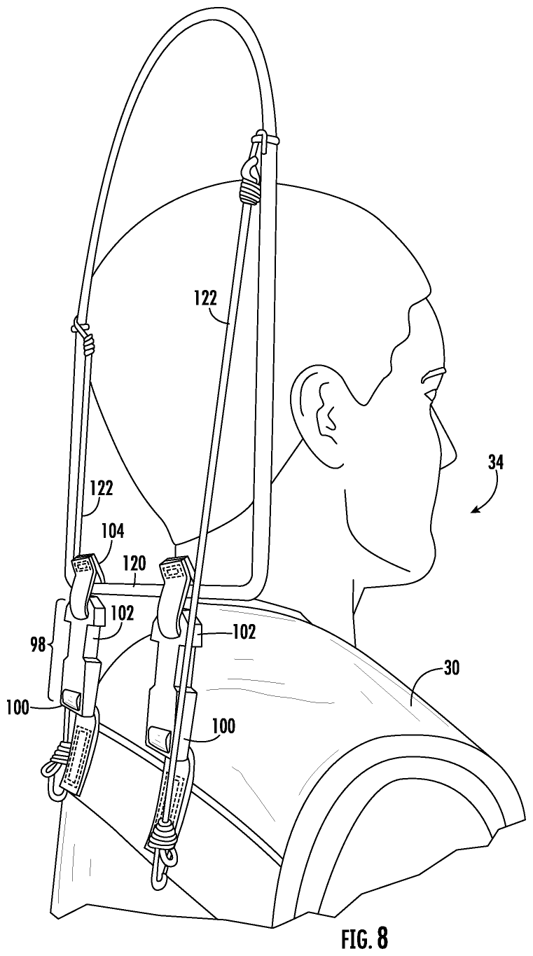

The hanger 32 is operable to suspend the personal radiation protection garment 30. The hanger 32 may be attached to the cable 28, the counter-balancer 26 or a means for connecting to the counter-balancer 26. The hanger 32 is sized so as to extend above the head of the operator 34 to avoid collision with the operator's head during manipulations. As shown in FIG. 5, the hanger 32 is configured so that it may be connected to the personal radiation protection garment 30. The hanger 32 may include an elongated rigid member 44 or bar bent in a “U” shape, including a curved portion 46 and side portions 48, 48′. Hooks 50 are provided at each end of the side portions 48, 48′ (FIG. 7). The hanger 32 is made of any material that can support at least a minimum weight of the suspended personal radiation protection garment 30. In one aspect, the hanger 32 can be a unified, rigid piece, such that the curved portion 46, side portions 48, 48′ and hooks 50 are integrated. The curved portion 46 is a portion of the hanger 32 bent into a “U” shape. The curved portion 46 is made of any material that can support at least a minimum weight of the suspended personal radiation protection garment 30. The curved portion 46 is meant to transverse around the top of the operator's head so that the curved portion 46 does not hit the operator's head while in use. The curved portion 46 has a first end and a second end.

In one embodiment, each side portion 48, 48′ of the bar 44 is aligned toward the shoulder sleeve of the personal radiation protection garment 30 close to the neck of the operator 34. This particular embodiment is effective at distributing weight and supporting the suspended garment 30. The hooks 50 of the hanger 32 may be located at the end of the side portions 48, 48′. Each hook 50 may be made of a material that can support at least a minimum weight of the suspended personal radiation protection garment 30. Each hook 50 is positioned over operator's shoulders. The suspended personal radiation protection garment 30 can be placed on the hooks 50, such that the hooks 50 support the weight of the garment 30. In one embodiment, the hooks 50 can be positioned slightly above the operator's shoulders, such that the hooks 50 act as a substitute for the operator's shoulders while the garment 30 is still substantially contoured to the operator's body.

As shown in FIGS. 5 and 7, a connecting system 54 may be provided for connecting the hanger 32 to the garment 30. The connecting system 54 is configured so that it may be connected to existing personal radiation protection garments 30. The connecting system 54 may have a buckle 56 and a strap 58 and is affixed to each hook 50 of the hanger 32. The connecting system 54 is operable for the hanger 32 to be connected to the garment 30, such that the connecting system 54 attaches the shoulder straps 55 of the garment 30 to the hanger 32. Two adjacent portions of strap 58 may be sewn together to form a small loop 65. The loop 65 is operable to connect to one of the hooks 50 of the hanger 32. The strap 58 forms a circle 63 when the two portions of the buckle 56 are attached to each other. The straps 58 are attached to the shoulder region of the garment 30 when on the garment 30. The distance between the straps 58 should be wide enough so that the head of the operator 34 can easily pass through the opening of the garment 30 and be clear of the straps 58. Each strap 58 may be threaded through the head opening and out of the arm opening of the garment 30. The buckles 58 secure the straps 58 in endless loops.

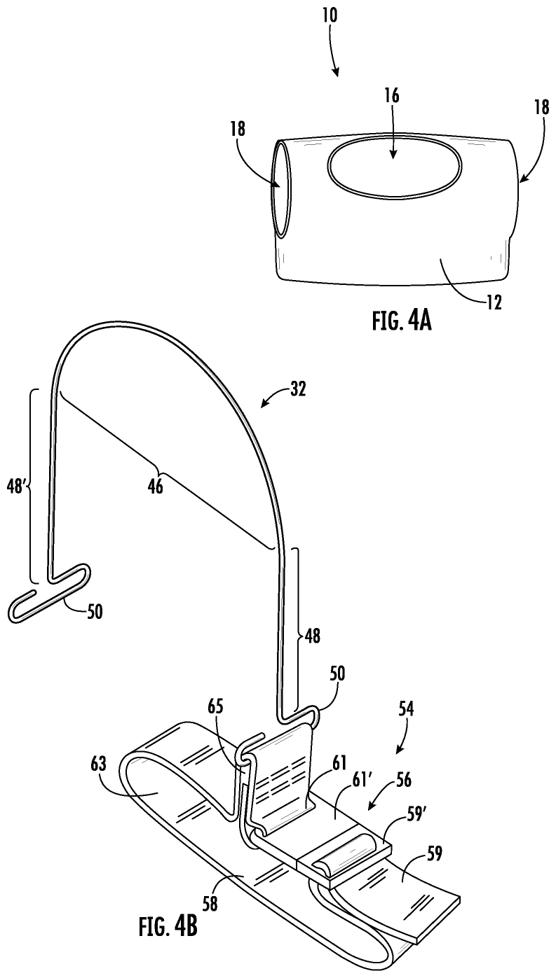

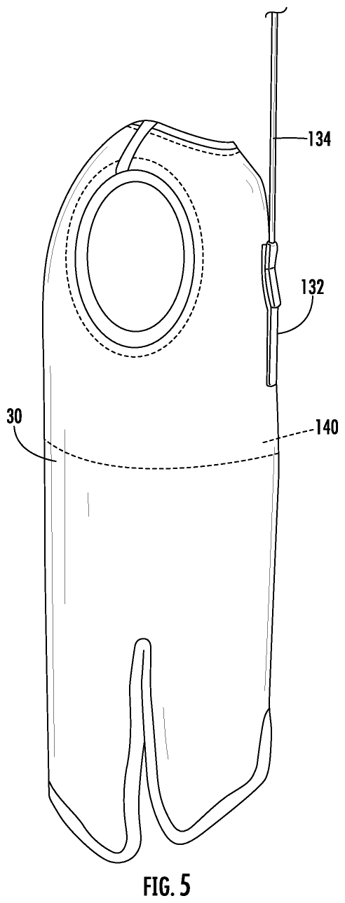

The personal radiation protection garment 30 includes a cushioning vest 10, an embodiment of which is shown in FIG. 4 and generally designated at 10. The vest 10 comprises a generally flexible body having a front portion 12 and a back portion 14. The vest 10 is open at the bottom for receiving the upper body of the medical operator 34. A neck opening 16 is provided for the head and an opening 18 is provided for each arm. The vest 10 may be inserted inside of the personal radiation protection garment 30 during manufacture. This material of the vest may be a foam or any other similar material that would conform to shoulders and back of a user for comfort, but also helps to cushion the space or the distance between the shoulders and the garment 30. For example, the connecting means may connect at the shoulder straps 55 as described above, wherein the cushioning material helps to maintain the spacing of the personal radiation protection garment 30 across the shoulders and back. This arrangement prevents the personal radiation protection garment 30 from falling when the garment 30 and the hanger 32 are released.

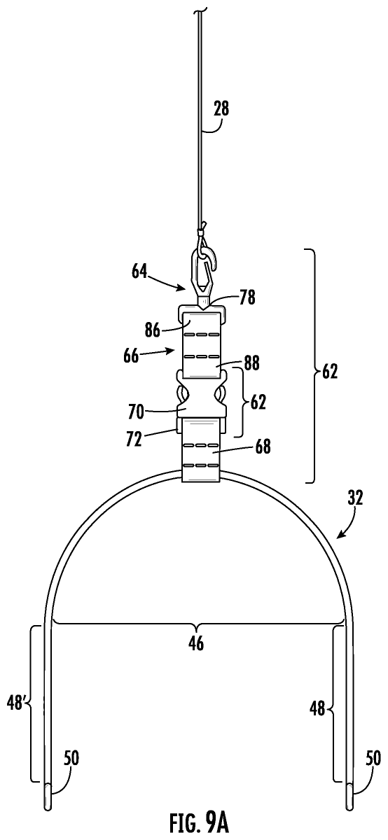

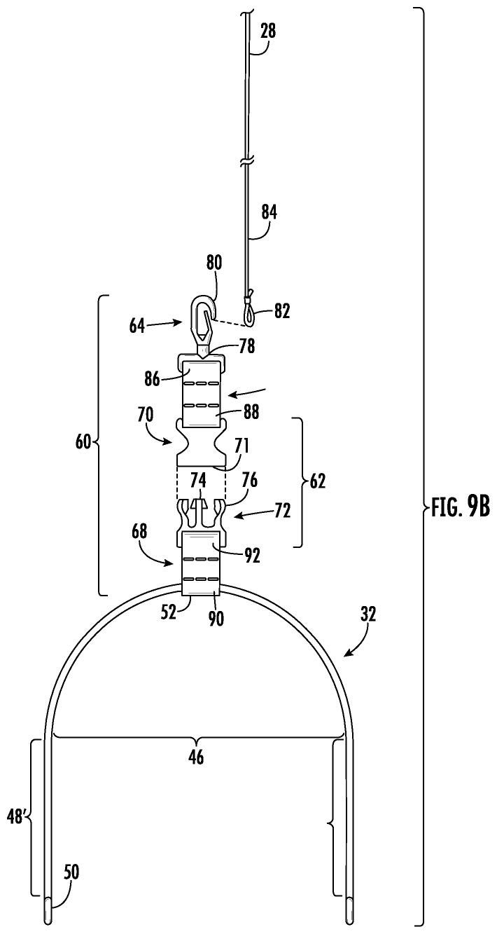

In one embodiment, the hanger 32 may be connected to the balancer 26 via a quick-connect means 60, as shown in FIGS. 9A and 9B. The quick-connect means 60 includes a buckle 62, a clip 64, a strap 66 connecting the buckle 62 to the clip and a strap 68 connecting the buckle 62 to the hanger 32. The buckle 62 of the quick-connect means 60 includes a first section 70 and a second section 72. The first buckle section 70 includes a receiving portion 71 and the second buckle section 72 includes an insertion portion 74 so that the second buckle section 72 is operable to removably connect to the first buckle section 70. The second buckle section 72 may have one or more depressible regions 76 that can quickly and easily be depressed to release the second buckle section 72 from the first buckle section 70. The buckle 62 may be made of material to support weight of suspended personal radiation protection garment 30. Both the first and second buckle sections 70, 72 have sections operable to receive the straps 66, 68.

The clip 64 of the quick-connect system 60 includes a base 78 and a fastening portion 80. The base 78 portion is operable to receive the strap 66 from the first buckle section 70. The fastening portion 80 of the clip 64 is operable to snap onto a loop 82 in the cable, a hook, or a rod. The fastening clip portion 80 may clip onto a portion 84 of the balancer 26 or clip onto the loop 82 of the cable or a strap (not shown) that is attached to the balancer 26. The clip 64 is capable of swiveling, that is the base 78 and the fastening portion 80 rotate relative to one another, so as to minimize twisting of the quick-connect means 60. The clip 64 may be made of material to support weight of suspended personal radiation protection garment 30.

The clip 64 and the buckle 62 may be connected via the strap 66. One end 86 of the strap 66 may be attached to the clip 64 and the other strap end 88 is attached to the buckle 62. Each respective end 86, 88 of the strap 66 is doubled back onto itself and sewn so as to be permanently attached. The buckle 62 may also be attached to the bar 44 of the hanger 32 by the other strap 68. The first end 90 of the strap 68 is connected to the bar 44 of the hanger 32 while the other end 92 of the strap 68 is connected to the buckle 62.

In accordance with an embodiment of the personal radiation protection garment suspension system 20, an exemplary method of operation is described below. The operator 34 of the suspension system 20 may present his personal radiation protection garment 30 to the suspension system 20. The personal radiation protection garment 30 may be any garment containing radiation-absorbing material. The personal radiation protection garment 30 may be of any shape, size or weight. The personal radiation protection garment 30 may be a full length protective garment, an apron, or a garment having any other amount of body coverage.

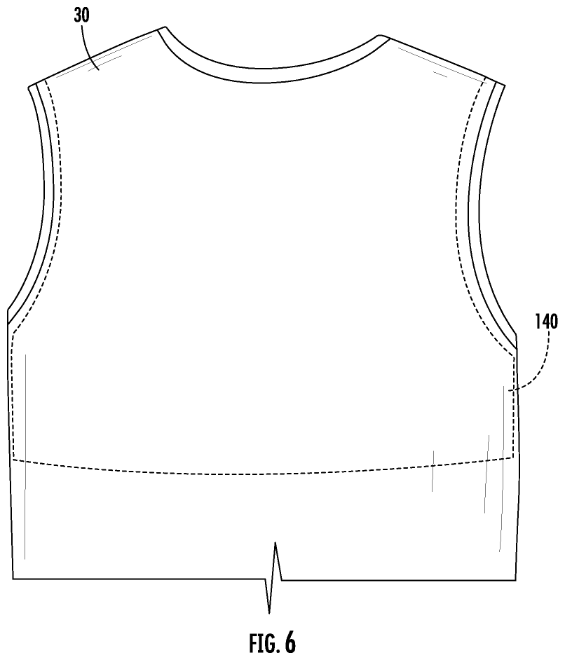

The hanger 32 may be pre-attached to the personal radiation protection garment 30 or to the suspension system 20. To attach the hanger 32 to the personal radiation protection garment 30, the connecting system 54 may be employed. As shown in FIG. 6, the shoulder area 55 of the personal radiation protection garment 30 connects to the connecting system 54 by looping the strap 58 of the connecting system 54 around the shoulder areas 55 of the garment 30 and buckling the two ends 61′, 59′ of the buckle 56 together. By doing this, the connecting system 54 effectively connects the hanger 32 to the personal radiation protection garment 30.

The operator 34 may attach the personal radiation protection garment 30 to the suspension system 20 by connecting the hanger 32 that has been pre-attached to the garment 30 to the suspension system 20, or attaching the garment 30 to the hanger 32 that has been pre-attached to the suspension system 20. The hanger 32 may be attached to the suspension system 20 using the quick-connect means 60, such as that previously described with respect to FIGS. 9A and 9B. The quick-connect means 60 acts as a means to quickly attach the personal radiation protection garment 30 to the suspension system 20 by connecting the ends 70, 72 of the buckle 62 together. The quick connect means 60 also acts as a means to quickly disconnect the garment 30 by depressing the insertion portion 74 of the buckle so that the ends 70, 72 of the buckle disconnect from each other.

After the personal radiation protection garment 30 is suspended from the suspension system 20, the operator 34 may step into the garment 30 and attach the garment 30 to the operator 34. For example, in order to secure the personal radiation protection garment 30 to the operator 34, the operator 34 may wrap the garment 30 around the operator's body and secure the garment 30 to itself by Velcro, buckles, or any suitable fastening means for attaching two pieces of a heavy material together. The operator 34 or another individual can strap the personal radiation protection garment 30 closed, such that the garment 30 is secured to the operator's body.

When operating with the suspension system 20, the operator 34 can move freely in the X-spatial plane and the Z-spatial plane while wearing the suspended personal radiation protection garment 30. The operator 34 can walk diagonally, crouch, or bend sideways in a free motion while receiving protection of the suspended personal radiation protection garment 30. The operator 34 may also have motion in the Y-spatial and the Z-spatial plane to use the radiation device to properly treat the patient 40. The suspended personal radiation protection garment 30 may be substantially weightless to the operator 34, such that the operator 34 is comfortable and unhindered. The arms of the operator 34 may be able to freely move in order to properly treat the patient 40. The operator 34 can bend over the patient 40 without causing pain to, or asserting extra weight on, the spine of the operator 34.

In accordance with the teachings of the present description, the suspension system 20 is operable to suspend the personal radiation protection garment 30 such that the operator 34 is not hindered or burdened by the weight from the garment 30. The suspension system 20 allows the operator 34 to have complete freedom of motion in the X-spatial plane and the Z-spatial plane, which is commonly used during medical and research procedures. For example, this allows the operator 34 to move laterally along the side of an operating surface 38, such as the table or a medical bed. Also, the operator 34 can freely perform vertical motion activities, such as stooping, leaning, squatting, standing on an elevated surface. The tension the balancer 26 asserts on the personal radiation protection garment 30 can be designed so as to provide optimum relief of the garment's weight for the operator 34. This force can be constant in all positions by the operator 34.

The operator 34 or another individual can quickly and effortlessly unfasten or simply quick-disconnect the personal radiation protection garment 30 from the suspension system 20 via the quick-connect means 60 in order to disconnect the garment 30 from the suspension system 20. The disconnecting point of the quick-connect means 60 may be at the counter-balancer 26, at the quick-connect means 60, or at the connecting system 54 connected to the hooks 50 of the hanger 32. Quick-disconnecting may be useful in emergency situations which occur during operating procedures. For example, it is not uncommon during a procedure in a cardiology catherization lab that a doctor must quickly move to another part of the room that is not covered by the suspension system 20, yet continue wearing the protective personal radiation protection garment 30. In this case, the quick-connect means 60 allows him to quickly disengage himself while wearing the protective personal radiation protection garment 30 from the suspension system 20, giving him full mobility about the radiation environment, such as having quick access the head or foot of the table, to perform various operations, such as intubating the patient or running a code.

It is understood that although an embodiment of a suspension system for a personal radiation protection garment is shown and described herein, the present apparatus may be used effectively regardless of the how the garment is suspended. The goal of the apparatus if protect the operator from sudden loss of suspension, whether intentional or even accidental. The quick-connect system 60 is described above as an exemplary system. It is to be understood that any other quick-connect system may be used to disconnect the hanger 32 or the personal radiation protection garment 30 from the suspension system 20. Examples of other quick-connect systems may include Velcro, clips, snap connectors, electronic connecting devices, fasteners, other connectors, and the like. In one embodiment, the quick-connect system 60 can be a quick release pin that joins cable sections 28. The pin is removed manually or remotely, when desired, using a solenoid to linearly actuate the solenoid. The solenoid provides the linear action instead of manually actuating the pin. Also suitable as a quick-connect system is a quarter-turn fastener, a quick release clamp or latch, or a quick-connect coupling made of metal or plastic. A rip cord may also be used in combination with, for example, a Velcro strap that is pull to actuate a latch.

Although the present invention has been shown and described in considerable detail with respect to only a few exemplary embodiments thereof, it should be understood by those skilled in the art that we do not intend to limit the invention to the embodiments since various modifications, omissions and additions may be made to the disclosed embodiments without materially departing from the novel teachings and advantages of the invention, particularly in light of the foregoing teachings. Accordingly, we intend to cover all such modifications, omission, additions and equivalents as may be included within the spirit and scope of the invention as defined by the following claims. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

I claim:

1. An apparatus for suspending a personal radiation protection garment above a work surface defining an area, the suspension apparatus comprising:
a support member adapted to be positioned above the work surface;
a cable mechanically suspended from the support member;
means for counter-balancing the weight of the personal radiation protection garment, the counter-balancing means attached to the cable; and
means for attaching the counter-balancing means to the personal radiation protection garment for suspending the personal radiation protection garment, the attaching means including means for disconnecting the garment from the counter-balancing means, the disconnecting means manually operable by one hand of a user and disposed within reach of the user while wearing the personal radiation protection garment attached to the counter-balancing means, wherein the personal radiation protection garment is supported in suspension when attached to the counter-balancing means, and wherein the personal radiation protection garment may be disconnected from the counter-balancing means by the one hand of the user for releasing the personal radiation protection garment from suspension while still being worn by the user.

2. The apparatus as recited in claim 1, wherein the counter-balancing means comprises a zero gravity balancer.

3. The apparatus as recited in claim 2, wherein the zero gravity balancer is operable to balance weight substantially equal to the weight of the personal radiation protection garment.

4. The apparatus as recited in claim 1, wherein the counter-balancing means comprises a spring balancer.

5. The apparatus as recited in claim 1, wherein the counter-balancing means comprises at least one counterweight.

6. The apparatus as recited in claim 1, wherein the counter-balancing means comprises an air balancer.

7. The apparatus as recited in claim 1, wherein the counter-balancing means comprises a pneumatic balancer.

8. The apparatus as recited in claim 1, wherein the counter-balancing means comprises a spring motor.

9. The apparatus as recited in claim 1, wherein the support member comprises a rail having a longitudinal axis; and a trolley disposed on the rail for movement relative to the rail along the longitudinal axis, wherein the cable is connected to the trolley.

10. The apparatus as recited in claim 9, wherein the rail comprises a means for attaching to the ceiling of a room.
